(19) United States
(12) Patent Application Publication
(10) Pub. No.: US 2026/0369376 A1
(43) Pub. Date: Nov. 19, 2026

(54) SYSTEM AND METHOD FOR HEALTH OPTIMIZATION AND MEDICATION ADHERANCE

(71) Applicant: WellnessIsMe LLC, Hoboken, NJ (US)

(72) Inventors: Arpita Hazra, Hoboken, NJ (US); Rupak Kumar Bag, Hoboken, NJ (US); Sudip Hazra, Santa Clara, CA (US)

(73) Assignee: WellnessIsMe LLC, Hoboken, NJ (US)

(21) Appl. No.: 19/548,793

(22) Filed: Feb. 24, 2026

Related U.S. Application Data
(60) Provisional application No. 63/805,280, filed on May 13, 2025.

(51) Int. Cl. G16H 20/13; G16H 40/63

(52) U.S. Cl. CPC G16H 20/13; G16H 40/63

(57) ABSTRACT

The present disclosure relates to a system for enhancing medication administration compliance through an integrated combination of software applications, an AI-enhanced medication management system, and a smart medication and/or supplement container. The system supports monitoring, recording, managing, and ensuring administration of medications in accordance with an established medication schedule defined by qualified sources such as patients and healthcare professionals. Medications include prescription drugs, over-the-counter medicines, supplements, and therapeutic substances in various dosage forms and release profiles. User interaction is enabled through multiple user devices and interfaces, including mobile devices, computers, wearables, and voice or gesture-based systems. Physiological and behavioral data from consumer and medical wearables may be incorporated to enrich health profiles and assess adherence and medication effects. The system may be used individually or in combination by patients, caregivers, and healthcare providers to improve adherence, oversight, and medication outcomes.

### CROSS-REFERENCE TO RELATED APPLICATIONS

The present application claims the benefit, under 35 U.S.C. § 119(e), to U.S. Provisional Patent Application No. 63/805,280, filed on May 13, 2025, the entire disclosure of which is incorporated by reference herein.

### BACKGROUND

Software applications for supporting medication adherence, whether accompanying pillboxes or operating as standalone solutions, typically provide features such as medication reminders, medication intake tracking, and monitoring of pillbox operation, among others. The information captured by such applications is often limited to data manually entered by the user or data derived solely from monitoring basic pillbox events. As a result, medication intake and adherence are frequently estimated in a coarse or incomplete manner. Moreover, the scope of captured information is generally limited and fails to account for additional factors, such as user behavior, contextual challenges, or barriers that may interfere with proper medication administration. Further, existing applications generally do not provide actionable insights to healthcare providers that would enable timely intervention or facilitate prediction of increased risk for missed or improper medication doses. Nor do existing applications leverage AI integration. This is not an exhaustive list but valuable opportunities to leverage available data to improve medication adherence, patient safety, and clinical outcomes are missed.

Containers adapted to organize, store, and dispense doses of medications (including pharmaceutical drugs as well as nutritional and therapeutic supplements) are widely used by patients to facilitate management of medication and supplement regimens. Such medications may be provided in various dosage forms, including but not limited to pills, capsules, tablets, powders, liquids, and gels, whether contained within a capsule or other suitable carrier. In some implementations, these containers are accompanied by software applications, such as mobile applications, configured to track medication intake and provide reminders. However, some patients are required to take multiple medications each day (potentially at different times, at varying frequencies, or in differing quantities) and ensuring proper administration in accordance with prescribed or optimal schedules remains challenging. This difficulty persists even when conventional tracking or reminder applications are employed. Existing applications typically rely on user-provided input or limited container-based data and operate as standalone systems, without providing access to healthcare providers the medication adherence information. As a result, incorrect or suboptimal medication adherence may go undetected, leading to delays in clinical intervention and missed opportunities for timely patient care.

Pillboxes, i.e., containers having several compartments such as, but not limited to, for each day of the week, for multiple weeks, etc., with or without labels and which facilitate organizing and medication doses, provide an easy way to monitor whether medication doses have been taken, and even provide reminders for overdue administration of medication, are very common and have been quite popular. More recently, further improvements and additional features have been provided with conventional pillboxes such as, but not limited to, locks for the entire container or one or more compartments, varied numbers and arrangements of dosage holding compartments, visual and audio alarms and other reminder mechanisms to facilitate medication adherence.

Nonetheless, conventional pillboxes typically involve a tradeoff between functionality and size or form factor, which often results in inadequate security features and limited medication schedule adherence monitoring, or conversely, increased size and complexity to accommodate such features. As used herein, “medication schedule adherence monitoring” refers to automatically determining whether a medication dose is accessed or dispensed in accordance with an intended dosing schedule, including identification of missed, delayed, early, or unauthorized doses. Many existing pillboxes rely on manually actuated lids or simple mechanical locking mechanisms, lack reliable user identification or authentication, and do not provide confirmation of authorized access or dispensing. These deficiencies can lead to unauthorized access, missed or incorrect dosing events, and reduced confidence in adherence tracking. Such risks are particularly significant in households with children, where inadvertent access to medications may result in hazardous or potentially life-threatening situations. Although some so-called “smart” pillboxes incorporate basic sensing capabilities, they frequently fail to integrate advanced security mechanisms, such as biometric authentication, together with precise dispensing feedback in a compact and portable form factor, thereby limiting their effectiveness in ensuring medication compliance, safety, and usability.

Despite the availability of software applications and pillbox-associated systems intended to support medication adherence, existing solutions generally lack an integrated artificial intelligence ecosystem capable of leveraging available data to meaningfully enhance adherence monitoring and clinical decision support. Current applications typically rely on manually entered information or limited container-based events and do not systematically analyze behavioral, contextual, or physiological factors that influence medication administration. As a result, adherence is often inferred in a coarse or incomplete manner, and patterns indicative of elevated risk for missed, delayed, or improper dosing frequently go undetected. Moreover, such systems commonly operate as standalone tools without advanced analytics, predictive modeling, or secure data sharing with healthcare providers, caregivers, or health insurance entities, thereby limiting opportunities for timely intervention, personalized support, and proactive care management. Consequently, valuable data that could be utilized through machine-learning and analytics techniques to improve medication adherence, patient safety, and clinical outcomes remains underutilized or unexploited.

The software application and smart pillbox disclosed and contemplated herein addresses and resolves the aforesaid shortcomings and provides several additional benefits over existing pillboxes and medication adherence software applications, as described in detail below.

### SUMMARY OF THE INVENTION

The present disclosure relates generally to systems and methods for medication management and adherence monitoring, and more particularly to a secure, portable medication container integrated within a data-driven medication adherence ecosystem.

In one aspect, the disclosed system and method provides a compact, portable pill container configured to store medication and control access thereto using biometric authentication. The biometric authentication mechanism is integrated into the pill container such that access to one or more medication compartments is permitted only upon successful biometric verification of an authorized user. The compact form factor enables personal portability while maintaining enhanced security relative to conventional pill containers.

In another aspect, the pill container is operable as part of a medication adherence data framework, wherein medication access events are detected and recorded. Such events may include, but are not limited to, the timing, frequency, and occurrence of medication removal. The pill container is configured to communicate collected data to a mobile computing device and/or a remote computing system.

In a further aspect, the system and method includes one or more analytics and machine learning modules configured to process medication adherence data collected from the pill container and associated software components. The analytics modules may analyze historical and real-time patient behavior related to medication usage and, in some embodiments, generate predictions regarding future medication adherence behavior.

In another aspect, the system is configured to generate personalized adherence support recommendations based on analyzed medication behavior. Such recommendations may include individualized interventions intended to improve adherence, including reminders, notifications, behavioral prompts, or other adherence-enhancing actions delivered through a software application or connected system.

In yet another aspect, the system and method enables secure transmission and selective sharing of medication adherence information with external systems. Medication behavior data collected from the pill container, a mobile application, and optionally one or more connected wearable or sensing devices may be transmitted to electronic health record systems, healthcare providers, care teams, or health insurance entities, subject to applicable authorization and privacy controls.

The disclosed systems and methods provide an integrated medication adherence solution combining biometric security, portable hardware, data collection, machine learning analytics, and interoperability with healthcare and insurance systems, thereby improving medication safety, adherence monitoring, and data-driven healthcare decision-making.

### SUMMARY

The present disclosure relates to a system for enhancing medication administration compliance. The system may include a combination of software application(s), an AI-enhanced medication management system (referring to the database, analytics and decision trees) and a smart medication and/or supplement container. In other embodiments, the container may further include a mechanized and electronic dispenser for dispensing a medication and/or supplement. These components of the system can be used in a combination or individually and provide features such as connectivity, and integration with external systems, facilitate monitoring, recording, managing, and ensuring administration of medications to a recipient in accordance with a predetermined schedule.

A system for health optimization and medication adherence includes a product container capable of holding one or more products for dispensing to an authorized user. The system may also include a biometric sensor sensing one or more biometric attributes to determine access to a product container compartment. In one implementation, the system may have a health optimization application tracking interactions with the product container to determine a medication adherence profile associated with the authorized user account and an adherence application transmitting one or more messages to the authorized user account. In some implementations, the system may include an authorized caregiver (a health professional, such as a nurse, home aide, and/or a family member or close friend) with permissions to interact with the product container in an effort to assist with medication adherence. The medication adherence profile will be focused on the authorized user or patient.

The product container may include a base, one or more product compartments and a lid. In one implementation, the one or more product compartments include one or more product sensors configured to sense whether a product is present in a given product compartment. The lid may include a locking mechanism and a lid sensor configured to sense if the lid is open or closed. The product container may include or be in communication with a processor and a memory in operable communication with a plurality of sensors including, the biometric sensor, compartment sensor and lid sensor; wherein the processor receives data from the plurality of sensors and stores the data in the memory.

The system for health optimization and medication adherence may include a number of data sources and/or databases. In one implementation, the system includes a HIPPA compliant database receiving, storing and transmitting electronic medical records associated with the authorized user. A user interactions database may be designed for receiving, storing and transmitting user interactions with the product dispenser. A user health database may also be configured for receiving, storing and transmitting user health data from one or more user devices. In one implementation, an enriched database may combine the HIPPA compliant database, the user interactions database and the user health database.

Software modules and/or applications may also be integrated with the system for health optimization and medication adherence. In one embodiment, a data analytics application may analyze information in the enriched database to determine adherence with a product schedule. A messaging application may be configured to transmit one or more messages based, at least in part, on an output received from the data analytics application. In one implementation, the one or more messages include an e-mail, a text message, an audio message, a visual message, a status light and a haptic feedback output. The system for health optimization and medication adherence may further include a user interface presenting the product schedule and the one or more messages.

A machine learning algorithm may be utilized for receiving data from the enriched database. In one implementation, the machine learning algorithm may develop a baseline behavioral profile for the authorized user, the baseline behavioral profile indicating a user's adherence to the product schedule. The machine learning algorithm may also analyze information stored in the enriched database and generate a predictive profile indicating likelihood of adherence. In one implementation, the machine learning algorithm generates one or more interventions designed to increase adherence to the product schedule.

The system may include generalized and/or tailored interventions, or a combination thereof. In one implementation, the interventions include a modification to the product schedule. The modifications may appear as suggested modifications targeted for physician/provider approval in an effort to improve medication adherence. The interventions may also include transmitting an intervention message to one or more user devices. For example, an intervention may send a message to a user's mobile phone, smart watch or smart glasses. Alternatively, the system may also send intervention messages to the product dispenser. In one embodiment, the system may also send an intervention message to one or more stakeholders.

In one implementation, tailored interventions may be patient-specific and dynamically generated. The system may generate individualized interventions based on analyzed adherence behavior and contextual data, including but not limited to. The interventions may include dynamically adjusting medication reminder timing, modality, or frequency based on predicted patient-specific adherence risk. The interventions may further include automatically notifying caregivers or providers when patient-specific nonadherence thresholds or risk patterns are detected. Additionally, some interventions may include delivering personalized behavioral nudges or engagement messages optimized to individual adherence patterns. In one implementation, the intervention may include triggering targeted pharmacist or care-team outreach when persistent or high-risk adherence gaps are predicted.

In certain embodiments, the system may provide generalized adherence support interventions across users. The generalized interventions may include scheduled medication reminder notifications based on prescribed dosing schedules. In one implementation, the system may transmit educational content related to medication adherence and chronic disease management through messaging applications, email, social network integration. The system may also provide routine adherence dashboards or reports accessible to users. Some system implementations may include automated refill reminders based on estimated medication supply duration. The generalized interventions may also include standard medication intake confirmation prompts following reminder delivery.

An established medication schedule may include, but is not limited to, a protocol which includes and specifies medication types, dosage amounts, dosage frequency, preferred or required times of day for medication administration, etc., as selected and determined by one or more qualified sources including but not limited to: a patient or recipient of medications, a physician, a nutritionist, a pharmacist, a medical professional, a medical expert, a health consultant, and other similarly qualified and knowledgeable persons.

A medication and/or medicine includes, but is not limited to pharmaceutical substances available by prescription, but rather include pharmaceutical substances, as well as over the counter medicines (e.g., acetaminophen, ibuprofen, antihistamines, digestive aids, allergy medicines, probiotic substances and supplements, etc.), therapeutic substances and supplements (herbal remedies and treatments, appetite suppressants and stimulants, etc.), nutritional substances and supplements (vitamins, minerals, etc.), and combinations thereof.

A dose and/or dosage may be used interchangeably to mean a quantity or amount of a medication which is being provided, or has been selected as being appropriate for inclusion in an established medication schedule, or is provided in a single standard or otherwise available dosage form. Generally, dosage forms typically susceptible to being organized, managed and monitored using the smart pillbox disclosed and contemplated herein include, but are not limited to, solid, powder, liquids, and gels, provided in any one or more dosage forms including: tablets, pills, capsules, powder, patches, gum, etc. (e.g., liquids and gels may be contained in a dosage form such as a capsule). Furthermore, dosage forms may be formulated in any of several release forms such as, but not limited to immediate release, delayed release, continuous or sustained release, soluble, dispersible, effervescent, chewable, and combinations thereof.

A user device may include any device which may be employed by a user to access, communicate, control, program, review, and otherwise interact with the smart pillbox and its various features, components, capabilities, and modules, etc. User devices include, for example without limitation, smart phones, laptops, tablets, wearable devices (e.g., without limitation-watches, glucose monitors, glasses), hardware based agentic assistants, computers, terminals, remote controllers, and the like.

A wearable may include any consumer or clinical-grade devices that passively collect physiological data and help enrich the patient's overall health profile. Examples include devices such as health watches, step trackers, sport watches, or smart bands that monitor heart rate, heart rate variability, pulse oximetry, sleep patterns, and daily activity levels. These data points help providers understand whether symptoms, like a persistently elevated heart rate, are related to lifestyle factors such as increased physical activity or may be associated with medication effects, for example with medications like Synthroid. The system may also include medical wearables such as continuous glucose monitors. These devices allow us to observe how a patient's diabetes medications are influencing blood glucose trends throughout the day. In general, any wearable that measures physiological parameters, vital signs, or behavioral patterns can serve as an important source of data enrichment, helping us build a more accurate picture of a patient's health and its association with medication adherence (captured by the product container and application software).

The User Interface is the part of a system, device, or software that allows a user to interact with it. It includes all the visual, auditory, and interactive elements through which a user gives instructions and receives feedback. The visual elements of the user interface may include Screens, windows, buttons, icons, menus, text, colors, layouts. The input methods may include a mouse, keyboard, touch, voice, gestures and/or controllers. The feedback may include alerts, messages, animations, sounds, status indicators and/or the like. In some implementations, the user interface may be embodied in one or more of the following: a graphical user interface, command line interface, touch interface, voice user interface and gesture based interface. A user may include a patient or other medication recipient, who uses the pillbox to organize, contain, and manage their medications, typically, but not necessarily, according to an established medication schedule, as well as others who may assist or guide the patient in the administration of their medication. Such others include, but are not limited to, friends, family members, care providers (e.g., home health aides), health care professionals (physicians, physicians'assistants), and other authorized medication dispensers and administrators.

Exemplary embodiments of the product container/smart pillbox are disclosed herein in detail. It should be understood that the disclosed embodiments are merely illustrative of the smart pillbox, the software application, and the operation of the AI-enhanced medication management system described and contemplated herein, each of which may be embodied in various forms. Any examples provided in connection with the described embodiments are intended to be illustrative and not limiting.

### BRIEF DESCRIPTION OF THE FIGURES

In order to more clearly illustrate the technical solutions in specific embodiments of the present application or in the prior art, the accompanying drawings to be used in the description of the specific embodiments or the prior art will be briefly introduced below, and apparently, the accompanying drawings in the following description are some of the embodiments of the present application, and for those skilled in the art, other drawings can be further obtained according to these drawings without any creative effort.

FIG. 1 is a product container according to an embodiment of the present application;

FIG. 2 is a product container according to an embodiment of the present application;

FIG. 3 is a product compartment of the product container according to an embodiment of the present application;

FIG. 4 is a diagram of one or more components of a health optimization and medication adherence system according to an embodiment of the present application;

FIG. 5 is a flow chart of a method for health optimization and medication adherence according to an embodiment of the present application;

FIG. 6 is a flow chart of a method for health optimization and medication adherence according to an embodiment of the present application;

FIG. 7 is a flow chart of a method for health optimization and medication adherence according to an embodiment of the present application;

FIG. 8 is a flow chart of a method for health optimization and medication adherence according to an embodiment of the present application;

FIG. 9 is a flow chart of a method for health optimization and medication adherence according to an embodiment of the present application;

FIG. 10 is a user interface for a system for health optimization and medication adherence according to an embodiment of the present application;

FIG. 11 is a user interface for a system for health optimization and medication adherence according to an embodiment of the present application;

FIG. 12 is a user interface for a system for health optimization and medication adherence according to an embodiment of the present application;

FIG. 13 is a network diagram for a system for health optimization and medication adherence according to an embodiment of the present application;

FIG. 14 is a network diagram for a system for health optimization and medication adherence according to an embodiment of the present application;

### DETAILED DESCRIPTION OF THE INVENTION

The present invention is described in the following examples, which are set forth to aid in the understanding of the invention, and should not be construed to limit in any way the scope of the invention as defined in the claims which follow thereafter.

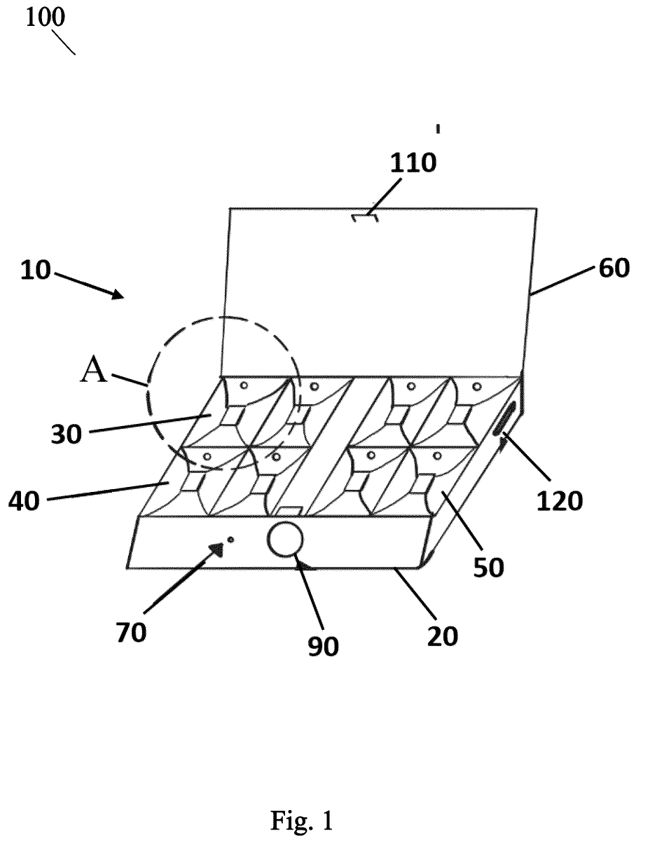

FIG. 1 is a product container according to an embodiment of the present application. More specifically, FIG. 1 provides a top-down perspective view of a first exemplary embodiment of a product container 10, also referred to as a smart pillbox 10, in its accessible open configuration. The pillbox 10 basically includes a pillbox body 20 with a plurality of compartments 30, 40, 50 and a lid 60, as shown in FIG. 1. The pillbox 10 also typically includes a microprocessor (not shown) for receiving, managing and transmitting or otherwise providing data, information, and instructions to and from various features of the pillbox 10, an externally located database of information, and an application (app) residing on or accessible through a user device, as described in further detail below. The smart pillbox comprises a container or pillbox body having a plurality of medication compartments, one or more locking mechanisms, a power management system, at least one microcontroller, a biometric authentication module, and a Bluetooth module for smart phone communication.

The presently disclosed and contemplated smart pillbox is a compact, portable, and complete system for medication organization, management and monitoring, which enhances and improves medication administration compliance with an established medication schedule. The smart pillbox includes a physical medication storage box as well as, for example, a fingerprint sensor for secure, child-proof, and tamper-resistant access. The pillbox integrates seamlessly with a Software/mobile application (mobile app), as well as other applications resident on or otherwise accessible through a user device (e.g., smart phone, tablet, etc.), via Bluetooth and/or WiFi, enabling secure real-time communication and data exchange, with external applications and systems including, but not limited to, Electronic Medical Record (EMR) systems, and Enterprise Management Systems (EMS).

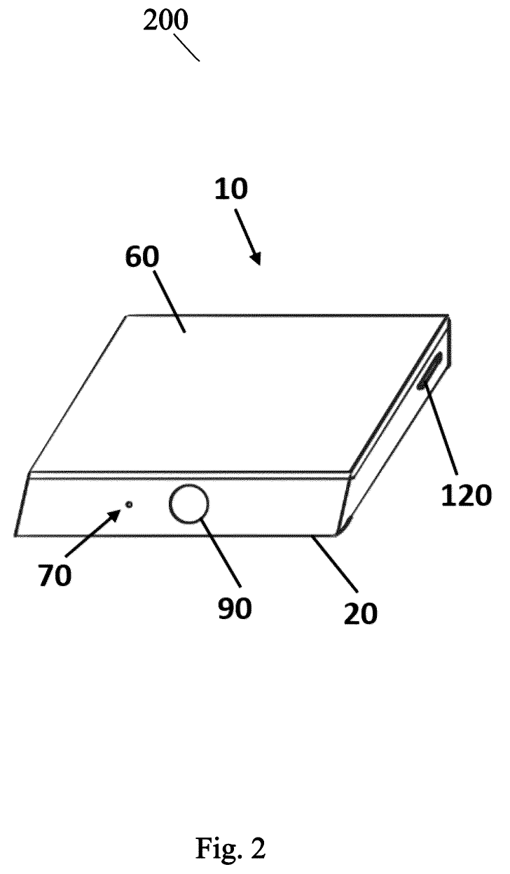

FIG. 2 is a product container according to an embodiment of the present application. More specifically, FIG. 2 provides a top-down perspective view of the pillbox 10 of FIG. 1 in its closed configuration, blocking access to the medication compartments 30, 40, 50 (not visible in FIG. 2, but see FIG. 1). The system may include medication reminders and alerts which includes sensors, programming, alert devices (e.g., audio (speakers), visual (LED lights), or a combination thereof), and an application residing on or accessible through a user device, all of which cooperate to enable the application along with the AI cloud based ecosystem to determine whether, when and what kind of reminders and alerts to issue to the user. The sensors monitor user behavior, such as the timing and frequency of medication access and administration, and communicate this user behavior information to the programming, the app, or both, for receiving, collecting, organizing, and analyzing the user behavior information.

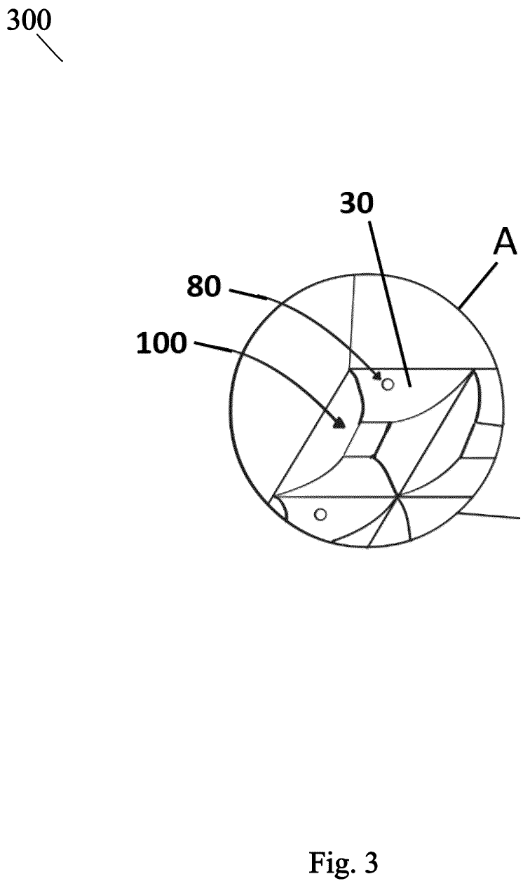

FIG. 3 is a product compartment of the product container according to an embodiment of the present application. More specifically, FIG. 3 shows a portion of the pillbox 10, encircled by a dotted line A in FIG. 1, in an enlarged perspective view to provide a clearer perspective view of one of the medication compartments 30 and features thereof, as described in further detail below.

Although the exemplary pillbox 10 shown in FIGS. 1-2 has a single lid 60 which is sized and shaped to cover and enclose all of the medication compartments 30, 40, 50, alternative embodiments are possible and contemplated in which, for example, the pillbox 10 includes a plurality of lids or lid segments (not shown), each of which covers and restricts access to one or more of the medication compartments 30, 40, 50. Moreover, the smart pillbox 10 may have a unitary construction (as shown in the figures), or may be manufactured as two or more components (not shown) which are assembled together, and may even be disassembled, such as for cleaning, replacement, etc.

In one implementation, the container may include a Light Indication System which illuminates the particular medication compartment which contains the particular medication to be taken (administered to the user/patient) at a particular scheduled time, according to the established medication schedule. This will help users easily identify which medication to take.

Additionally, the pillbox 10 may advantageously include a low energy Bluetooth (BLE) module which enables communication with an application resident and running on the user device (e.g., phone, tablet, PC, agentic assistant) which can then provide real-time notifications and alerts to the user for low battery, full charge, medication adherence, and refill requirements, via the app. For example, the Bluetooth device or module may transmit user adherence information or data (e.g., pill removal, compartment status) to the app, notifying users of dispensing events or refill needs.

Other useful features which may be provided with the pillbox 10 include those which detect and report whether the pillbox 10 is in an open or closed state. For example, magnetic sensors such as a reed switch, can detect and report whether the open or closed state of the pillbox 10, and trigger appropriate system responses as programmed by a user. Additionally, auditory feedback may be provided by the pillbox 10 using a buzzer which issues alerts for scheduled medication times, and is configurable to stop upon pillbox opening or after a set number of alerts as would be programmed by a user.

With continued reference to FIGS. 1-3, the microprocessor (not shown) of the pillbox 10 receives, manages, analyzes, and transmits or otherwise provides data, information, and instructions to and from features on the pillbox 10, such as visual (e.g., light emitting diodes (LEDs) 70, 80) and audio (e.g., speakers, not shown) signal devices, and sensors (e.g., biometric sensors, such as fingerprint sensors 90, presence sensors, such as non-contact sensors, and infrared-based detectors 100). For example, visual indicators, such as LEDs 80 positioned in each of the compartments 30, 40, 50, may include brightness control for visibility and may usefully highlight the medication compartment containing the scheduled medication. If a compartment is empty and scheduled for a dose, the LED 80 may, for example, illuminate red to indicate the need for refilling with new medication dose(s). Non-contact sensors, such as infrared-based detectors 100 (see FIG. 3), monitor for and detect the presence or absence of medication in the compartments 30, 40, 50, providing feedback to the microcontroller and, via Bluetooth, the microcontroller, or a combination thereof, to the app residing on a user device.

Various features of the pillbox 10, such as those described above, collect and transmit to a database or app, several types of information and data including, but is not limited to, details of user (patient) behavior such as time and day of access to, and administration of, medication, as well as biometric information regarding users gaining access to the compartments 30, 40, 50 of the pillbox 10, such as the identity of such users and the day and time of such access.

The smart pillbox 10 described and contemplated herein may further comprise a locking system which includes one or more locking mechanisms 110 (see FIG. 1) corresponding to and capable of locking the one or more lids 60 in a position which encloses and prevents access to one or more corresponding medication compartments 30, 40, 50 (see FIG. 2). Furthermore, the locking system includes programming which electronically communicates, via the microprocessor (not shown), with and controls the one or more locking mechanisms 110, based on communications with and instructions from a user interface such as may be resident on a user device (not shown).

The smart pillbox 10 also comprises a power source and management system which may, for example, include a rechargeable lithium polymer battery and a battery management system for controlled charging. An externally accessible charging interface, such as a USB-C port 120 (see FIGS. 1-2), receives external power for charging the battery which is protected by overcurrent and surge protection features. A green LED 70 is provided on an outer surface of the pillbox body 20 for indicating charging status of the battery. Although not shown, per se, a red LED may also be provided on the pillbox body 20 for illuminating when the battery is low, signaling the need for recharging. In one implementation, the LED or light indication system may be compartment specific. In other words, each compartment may have an associated LED that may be configured to turn on when a medication is scheduled to be administered based on a medication schedule. In other embodiments, a wearable may alert the user that one or more biomarkers is out of range and suggest a product stored in the product dispenser. For example, if a glucose monitor alerts for high blood sugar, the system may suggest one or more supplements including, but not limited to berberine, cinnamon, chromium, magnesium, and alpha-lipoic acid. Conversely, if a glucose monitor alerts for low blood sugar, the system may suggest a glucose tablet.

Furthermore, the power source and management system of the pillbox 10 may include app-based alerts to provide, monitor and ensure reliable power management and availability, making the device suitable for portable healthcare applications. A power regulation module may also be included for delivering stable voltage outputs to the microcontroller and peripherals and maintaining operation across varying battery states. A voltage measurement circuit may be included with the pillbox 10 for periodically assessing battery status, selectively activating to minimize power consumption. Furthermore, battery charge levels may be periodically transmitted, via Bluetooth, the microprocessor or a combination thereof, to an app which is resident on a user device, which then displays low battery notifications and alerts users when the device is fully charged.

In one embodiment, the product container/smart pillbox is a medication storage container configured to store pharmaceutical substances in a safe and secure manner and to facilitate medication adherence. In some embodiments, the smart pillbox comprises one or more sensors, actuators, and light-emitting indicators configured to provide features including, but not limited to, biometric locking, a plurality of medication storage compartments (e.g., eight pill storage slots) with indicator-based identification, and pill-presence detection. The smart pillbox may further include an onboard power source, such as a rechargeable battery, and a controller or microcontroller configured to communicate with external devices via one or more wireless communication protocols, including Bluetooth and Wi-Fi. The smart pillbox may include a lid covering the entire container and/or individual lids corresponding to respective medication compartments. In some embodiments, the smart pillbox is rechargeable via a universal serial bus (USB-C) interface and includes one or more locking mechanisms configured to restrict access to authorized users. The smart pillbox may further be configured to store and process biometric data, such as fingerprint data, for user authentication. Additionally, the container may include sensors configured to detect opening and closing events of the lid and one or more audio output devices, such as a buzzer, configured to provide audible alerts.

In some embodiments, enabling the dispensing of medication to an authorized user by the product container may, for example, follow a method. First, upon detecting an open pillbox or medication compartment (e.g., via magnetic sensors; not shown) the microcontroller may activate one or more LEDs to indicate the particular compartment containing the scheduled medication to be dispensed according to an established medication schedule previously provided to the app or microcontroller. Infrared sensors or other types of sensors provided in each medication compartment may be configured to monitor and detect the presence or absence of medication in the respective compartments. The status of patient/user adherence to the established medication schedule may be reported and transmitted to the application software. If a particular medication compartment is empty, an LED provided in that compartment may illuminate red, a notification may be sent to the app, or both, to alert the user to refill that compartment. At scheduled medication dispensing (administration) times, auditory alerts may activate until the pillbox is opened or a set number of alerts is reached, with corresponding app notifications optionally also being provided. Furthermore, upon closure of the pillbox, indicators and sensors may deactivate, which allows the pillbox and its modules and systems to enter low-power mode after a delay, with the fingerprint sensor active in standby, in preparation for the next cycle. The sequence of operations is presented as exemplary while other sequences may be implemented without departing from the scope of the disclosed techniques.

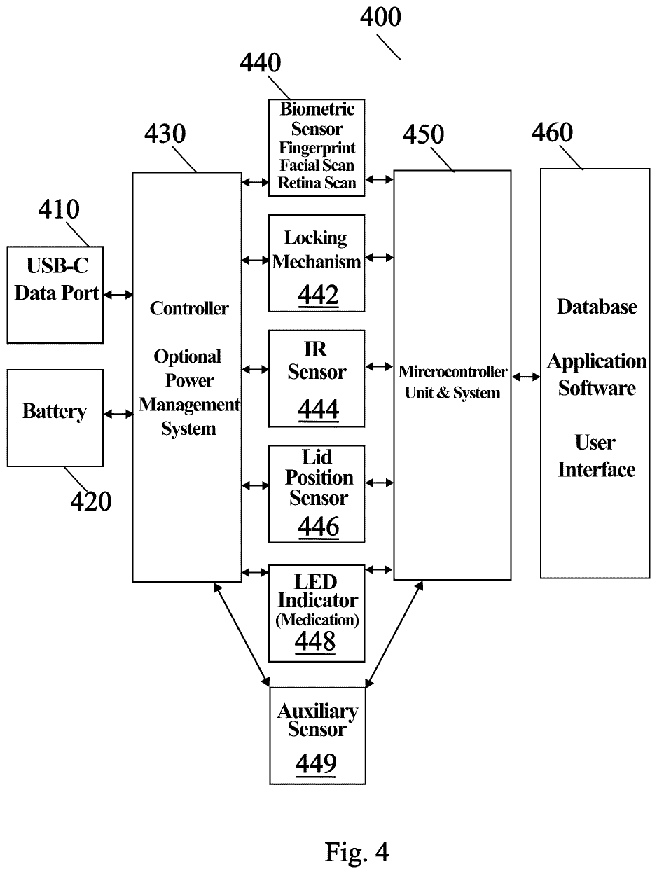

FIG. 4 is a diagram of one or more components of a health optimization and medication adherence system according to an embodiment of the present application. The illustrated system 400 includes a USB-C data port 410, a battery/power source 420, a controller/optional power management system 430, a biometric sensor 440, a locking mechanism 442, an infrared (IR) sensor 444, a lid position sensor 446, an LED indicator 448, a microcontroller unit and system 450 and a database, application software and user interface component 460.

An auxiliary sensor 449 may include one or more sensors to support the functionality of the system and may be integrated into the product container without departing from the scope of the claimed techniques. For example, an auxiliary sensor may include one or more motion and position sensors which may be integrated to detect movement inside the container indicating consumption of a product, medication and/or supplement. These motion and position sensors may include an accelerometer, gyroscope, and the like. Optical, proximity and laser sensors can be used alone or in combination with the infrared sensor for detecting the presence, quantity and or difference in the quantity of pills before and after a lid open/close event. An RFID sensor may be utilized to track medication inventory and ensure medication has not expired. Pressure and force sensors may be used to detect when a compartment or container is being accessed. Environmental sensors may be utilized to detect temperature and humidity over time and for predetermined intervals to ensure medication stability and efficacy. For example, if a container is left in an environment with a temperature greater than 80 degrees Fahrenheit for more than six hours, the container may alert the patient/user/caregiver that the medication is no longer considered effective. Different medications may have distinct temperature and humidity thresholds over different temporal exposure intervals.

The biometric sensor 440 includes sensing and electronic communications apparatus which perform fingerprint sensing and recognition, as well as encrypted storage and anti-spoofing features to restrict access to unauthorized users, offering peace of mind by ensuring that no one else, particularly children, can access the medication, preventing dangerous incidents. In one embodiment, the biometric sensor/authentication module may control access to medication doses disposed in the medication compartments of the pillbox body based on sensing and confirming biometric features (e.g., fingerprints, retinal scans, voice prints, facial features, etc.) of authorized users of the pillbox. The biometric authentication module includes sensors and programming to detect, read, and determine whether a biometric attribute of a user is authorized to access the medication compartments, as well to communicate permission to release a locking mechanism on the pillbox body which otherwise prevents access to the medication compartments.

The biometric authentication system provides peace of mind by ensuring that no unauthorized individuals, especially children in the household, can access the medication, preventing potentially perilous situations such as accidental ingestion. The biometric authentication module also minimizes or prevents misuse and theft of medication in the pillbox, which are particularly important and useful for controlled substances, such as opioids and other pain control medications, steroids, ADHD medications and anti-depressants, etc.

In some embodiments, the biometric authentication module includes a high-sensitivity biometric sensor which captures fingerprint data (e.g., the fingerprint sensor 90 shown in FIGS. 1-2) for user authentication, operating in a low-power standby mode until activated by touch. In some embodiments, the biometric sensor, whether fingerprint sensor 90 or other type of sensor (e.g., retinal scanning), remains in standby, consuming minimal power until a finger touch is detected, triggering the microcontroller to exit low-power mode and initiate authentication, regardless of fingerprint recognition outcome. Upon finger detection, the fingerprint sensor 90 may activate and signal the microcontroller (not shown) to wake, and capture fingerprint data. The microcontroller may then verify the data against encrypted templates using a secure matching algorithm. In some embodiments, for example, successful fingerprint (or other biometric) authentication may unlock the pillbox 10 for a short time, e.g., 5 or 10 seconds, after which the lock re-engages automatically, requiring the user to open the pillbox within this window. Unsuccessful attempts are managed per predefined security protocols, protecting against unauthorized access.

Fingerprint templates specific to and for identification of authorized users may, for example, be stored in a secure memory module, or an externally accessible database, which are protected by cryptographic techniques. Use of fingerprint identification prevents unauthorized access or tampering, ensuring only authorized users can unlock the pillbox. The biometric authentication module is capable of and supports enrollment and authentication of multiple users (e.g., patients and caregivers), by storing individual profiles securely to accommodate shared use while maintaining strict access control. The fingerprint sensor 90 may employ liveness detection to differentiate genuine fingerprints from artificial replicas (e.g., silicone molds or printed images), which further enhances security by preventing spoofing attempts that could compromise medication safety. Authentication attempts may be limited by the biometric authentication module to prevent brute-force attacks, with failed attempts triggering configurable responses, such as temporary lockout or auditory alerts. Authentication events are logged for auditing, ensuring traceability. Failed authentication may trigger limited retries, followed by configurable security measures (e.g., lockout, alerts).

In one embodiment, the biometric sensor/authentication module may require two or more forms of biometric authentication for access and/or entry after an enforced lockout. For example, the biometric sensor/authentication module may require a fingerprint and facial recognition scan, a fingerprint and audio voice matching, a fingerprint and retinal scan or any permutation thereof. In fact, the biometric sensor can be uniquely adapted to guard against AI generated cloned biometrics like deepfake audio recordings and facial images and/or the like. Additionally, the biometric sensor may also be combined with a multi-factor authentication approach requiring either a password, a one time passcode sent via text message, a push notification sent to a user device requiring approval at the user device.

The microcontroller 450 coordinates and controls the condition of, access to, and user notification regarding the medication compartments using sensors, indicators, and locking mechanisms, to provide visual and auditory alerts and notifications on the pillbox itself, as well as via user devices including, for example, smart phones, tablets, etc., to guide and encourage medication intake and monitor user adherence to an established medication schedule or protocol. The smart pillbox also includes a battery 420 (e.g., lithium polymer), status indicating light emitting diode (LED) 448 and app-based alerts to ensure reliable power availability and management, making the smart pillbox suitable for and capable of portable healthcare applications.

The system for health optimization and medication adherence may include other sensors, e.g., a compartment sensor detecting whether medication exists in a particular compartment. The compartment sensor may be implemented via an infrared sensor and may trigger an alert sent to the user to refill the compartment. The system may further include a lid position sensor 446 detecting whether the lid is in an open or closed position. For example, the time when the medication compartment is opened (accessed) may be sensed and then transmitted to the app for recording and tracking compliance with an established medication schedule. Additionally, other collection devices may be integrated for monitoring and collecting user behavior information, such as the timing and frequency of medication compartment access, and communicating such user behavior information to programming of a microprocessor provided with the smart pillbox or an app which resides on or is accessed through a user device.

The application software 460 may provide access to an AI-enhanced medication management system. The software application may be installed on a smartphone, wearable device, tablet, computer, or other computing device capable of connecting to the Internet via a wired or wireless communication interface in order to access the AI-enhanced medication management system. The software application is configured to communicate with a pillbox or other compatible container adapted for medication storage via one or more wireless communication protocols, including but not limited to Bluetooth or Wi-Fi, to record information associated with opening and closing events of the pillbox or compatible container and to track medication or supplement consumption, including medication placement and removal.

The pillbox or compatible container may include one or more sensors, light-emitting indicators, and actuators configured to provide features such as biometric locking, indicator-based identification, and pill-presence detection, each of which may be monitored and controlled via the software application. The software application further comprises a user interface configured to capture, store, and manage medication management data, including organization of medications according to planned intake schedules, patient medication adherence behavior (including schedule compliance, on-time intake, missed doses, and delayed intake), and patient-reported symptoms; to receive and display medication intake monitoring data from the secure pillbox; and to transmit captured data to the AI-enhanced medication management system. In some embodiments, the software application is further configured to capture or receive data from one or more additional health monitoring applications installed on the smartphone, tablet, or computer.

The AI-enhanced medication management system may be implemented on a cloud-based computing infrastructure and is configured to analyze captured data, predict medication-related behavior, and correlate patient-reported symptoms with medication adherence patterns using integrated data from wearable devices. The system may further enhance collected data by incorporating physiological parameters obtained from connected consumer-grade or clinical-grade wearable devices, including but not limited to heart rate, heart rate variability, sleep patterns, activity levels, and blood glucose trends. In some embodiments, the AI-enhanced medication management system is configured to generate individualized interventions derived from decision trees or other machine-learning techniques based on analyzed data and patient lifestyle patterns, including daily routines and habits that influence optimal medication timing.

Machine learning algorithms that may be implemented include predictive analytics approaches such as logistic regression, random forest, and gradient boosting models to analyze medication adherence patterns, along with time-series models to understand behavioral trends over time. These models can leverage inputs such as medication usage patterns, wearable activity data, and historical adherence behavior to improve reminder timing and user engagement. The models are intended to support adherence insights and application functionality.

In one embodiment, outputs of the machine learning algorithms may include predictive algorithms for a patient's health in relation to medication adherence and potential medication side effects. The output may also map patient behavioral attributes that influence medication adherence. The machine learning techniques may be utilized to develop tailored tools to support medication adherence. For example, providing a virtual nurse to assist some patients with medication setup in the device and app, or sharing adherence insights with providers to enable solutions customized to individual patient needs.

The machine learning algorithms may be utilized to support providers in identifying possible root causes of patient symptoms, such as missed blood pressure medications over multiple days contributing to adverse events like stroke, etc. This can combine captured adherence and behavioral data with information from EMRs, wearables, glucose monitors, and other health-tracking devices to enable patient-centered, tailored interventions.

Interventions may be based on patient lifestyle and analysis of medication adherence data. Patient lifestyle refers to day-to-day routines or habits that can influence the best time for taking certain medications. For example, a patient who regularly exercises in the early evening may experience a naturally higher heart rate at that time, which could make it less ideal for taking a medication that also increases heart rate. Another example is a patient who consistently works late on certain weekdays or attends social events where they consume alcohol, which may cause them to forget or intentionally skip an evening dose. Understanding these patterns helps clinicians adjust medication timing so it fits naturally into the patient's lifestyle and improves adherence.

The system may further be configured to share medication behavior information with electronic health record systems, healthcare providers, caregivers, and health insurance entities, and to present AI-generated insights, predictions, recommended interventions, and adherence feedback to the user via the software application. The cloud-based AI system may comprise one or more databases for storing data captured from the software application and the pillbox, and one or more machine-learning models configured to analyze, predict, correlate, enhance, and transmit patient medication-related behavior data.

The software application, smart pillbox, and AI-enhanced medication management system, when used together as an integrated system, in partial combination, or individually as described herein, provides assistance to users including, but not limited to, patients, caregivers, healthcare professionals, and other authorized medication dispensers or administrators, and facilitate organizing, managing, and monitoring one or more medication doses in accordance with an established medication administration schedule. In one implementation, an artificial intelligence medication adherence predictive algorithm may analyze user behavior user information to predict when a user is likely to miss a medication dose and preemptively provides notification to the user, a caregiver, or both in advance of the time for administration of the medication dose. A non-compliance remediation algorithm may include computer instructions, that when executed, cause a software application or module, or a combination thereof, to provide remediation features and notifications to one or more of: the user's friends, the user's family members, and a care provider, to prompt a home check or other action to assist the user with medication administration.

The software application, smart pillbox, and AI-enhanced medication management system, whether used together, in partial combination, or individually, generally include features and capabilities for connectivity, integration, and interaction with external systems, applications, and data sources, and facilitate monitoring, data capture, database storage, recording, management, and administration of medications to a recipient in accordance with an established medication schedule. The software application may include a gamified adherence program to engage users and provide a behavioral reinforcement system integrated with physical medication control. A gamified adherence program may include a user interface, a points system, levels of progression, badges or achievements, challenges or missions, and rewards. The points may and/or rewards may be convertible to virtual currencies, virtual goods, and/or real life goods.

The software application, smart pillbox, and AI-enhanced medication management system, whether used together, in partial combination, or individually as described herein, enhance user health, safety, and welfare in personal healthcare, assisted living, and clinical environments by providing a secure and portable solution for medication management. These components collectively address objectives including, but not limited to, patient safety, medication adherence monitoring, and prevention of unauthorized access to medications, particularly by children, across diverse usage environments.

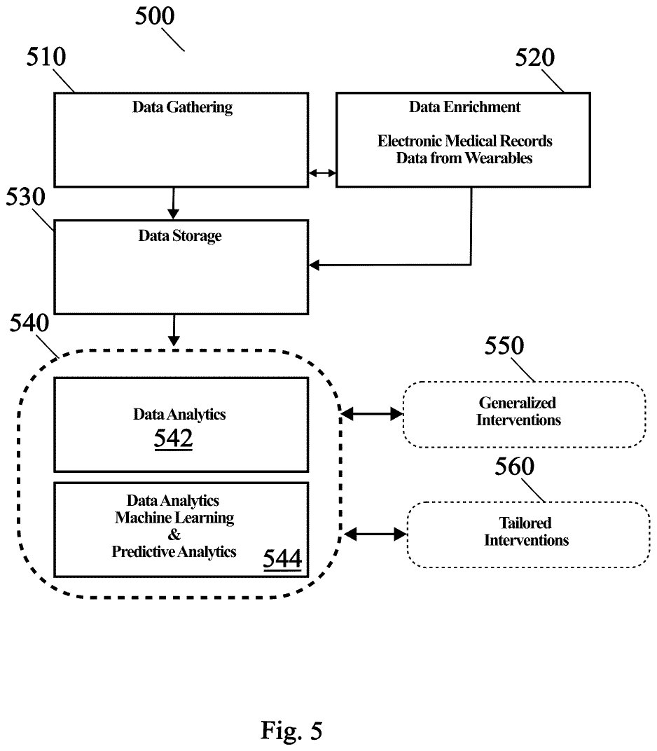

FIG. 5 is a flow chart of a method for health optimization and medication adherence according to an embodiment of the present application. The flow chart 500 includes a data gathering step 510, a data enrichment step 520, a data storage step 530, a data analytics step 540 involving data analytics in real time 542 and data analytics with machine learning and predictive analytics 544. The flow chart 500 may further include one or more outputs. For example, the system may output generalized interventions 550 and/or tailored interventions 560.

In one embodiment, the data gathering may receive data from the product container/smart pillbox, a user device and/or application software. For example, the data received from the product container may include timestamps associated with compartment access and sensor data indicating the presence or absence of medication within the compartment. The data enrichment step 520 may combine or enrich data received from the product container with data received from external (external to the product dispenser) sources. For example, data received from user devices such as wearables as well as electronic medical records may be used to enrich the data from the smart pillbox and vice versa.

In one embodiment, the system acquires data analytics 542, for example, acquiring real-time behavioral data from the product container to identify when and what medications are consumed by the user. The data analytics may be acquired in real time, over a predetermined temporal interval, for example, hourly, daily, weekly, monthly. Additionally, the interval may be based on medication start dates, titration schedules and the like. This data may be processed to determine a user behavioral baseline. The data analytics with machine learning and predictive analytics 544 may utilize the user behavioral baseline to provide user predictions over a time horizon or interval. In one embodiment, the machine learning and predictive analytics may generate a risk score or risk profile based on a combination of the data analytics data. The risk score and/or profile may then inform when and what kind of interventions should be triggered.

The system may offer generalized interventions 550. For example, a patient nudge may be implemented as an LED blinking light on the dispenser/pill box suggesting consumption of a medication is upcoming, due or missed/overdue. Another generalized intervention may include a reminder sent via the application software to a user device similarly indicating consumption of a medication is upcoming, due or missed/overdue. The tailored interventions 560 may assist and/or reinforce medication adherence by suggesting a modification to medication timing to align with a user's behavior patterns revealed with the enriched data set.

Data enrichment may further include leveraging an AI-enhanced medication management ecosystem comprising a cloud-based AI system communicatively coupled to a mobile application (executable on a mobile device) and a secure product container device comprising a biometric lock, eight pill storage slots with LED-based identification, and pill presence detection sensors. The cloud-based AI system includes data enhancement capabilities that actively integrate, enrich, and augment medication adherence data. The medication adherence data may be captured from the mobile/software application (including pill management schedules, adherence behavior such as on-time/missed/late intake, and patient-reported symptoms) and the secure product container (real-time pill intake monitoring), with additional physiological, health-related, and contextual parameters sourced from connected consumer or clinical-grade wearables, medical devices, electronic health records, or other authorized external systems. The wearables may capture data, including but not limited to, heart rate, heart rate variability, pulse oximetry, sleep patterns, daily activity levels, blood glucose trends from continuous glucose monitors, lifestyle-derived insights, and relevant clinical data.

The enhanced and augmented dataset may be continuously stored and updated in a cloud-based database such as a cloud-based data lake. With this stored data, machine learning models may perform deeper, more accurate analysis, correlation of patient symptoms with medication adherence and behaviors, prediction of medication-related patterns, identification of causal associations (e.g., elevated heart rate or glucose spikes linked to missed doses), and generation of highly individualized, context-aware interventions derived from decision trees, incorporating comprehensive patient health profiles, lifestyle factors, physiological responses, and medication effects to significantly improve adherence, clinical outcomes, and personalized care delivery. In one implementation, a machine learning algorithm may analyze patient medication related behavior (patient taking medication on time, missing medication, taking medicine late etc.). The machine learning algorithm may predict future medication adherence behavior (e.g., a patient is most likely to miss medications if scheduled at noon). This may assist the physician to modify the schedule of medications to increase likelihood of compliance. Machine learning may also analyze patient symptoms (entered via application software and/or captured with other user devices) and its correlation with patient's medication and medication related behaviors. (e.g. patient has a high heart rate on days he misses medicine X, medicine X missed data may be captured by the product container and/or user device). In one implementation, a wearable may capture a high heart rate and through cloud based data enhancement, the information may be integrated with patient medication and behavioral data. The integrated information may also be transmitted to stakeholders. In one embodiment, the integrated information may provide insights to providers who can suggest interventions to assist patient medication compliance based on decision trees.

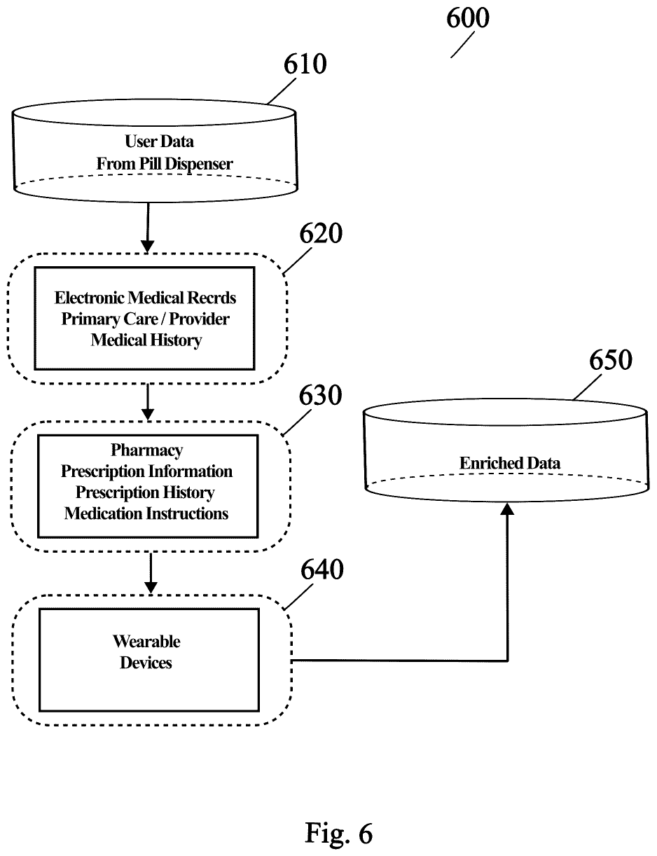

FIG. 6 is a flow chart of a method for health optimization and medication adherence according to an embodiment of the present application. The flow chart 600 includes an original data source of user data from a product container/smart pillbox 610. The user data may include information indicative of adherence to a medication schedule, user behaviors and/or vital statistics. The data source may be combined with electronic medical records from a medical provider including a primary care physician, medical specialists, medical history and/or the like 620. Other data sources such as pharmacy data, prescription information, prescription history and medication instructions 630 may also be combined with the original data source. Pharmacy and prescription information may also include prescription drug names and dosage information. Wearable devices may provide wearable user data 640 indicating physical activity, pulse, resting heart rate, blood oxygen readings, EKG readings, glucose readings and the like.

Optionally, the system may utilize the prescription information received to obtain scientific studies and medical journals discussing the prescriptions being taken by the user. A machine learning model may then take the corpus of scientific studies and medical journals and identify a set of commonly associated side effects, interactions and results from these studies. With this information, the system may then present a user with a targeted prompt inquiring about the associated side effects and/or potential interactions with other substances.

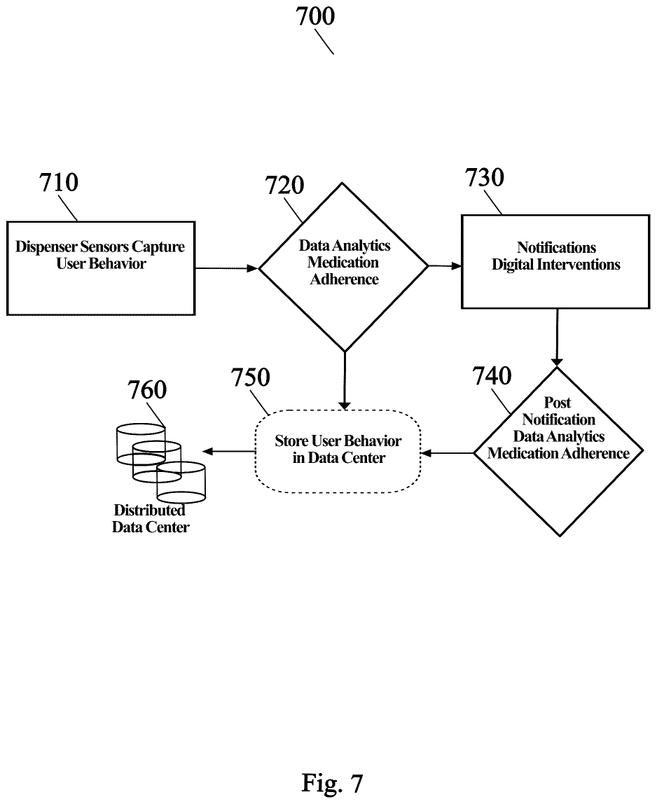

FIG. 7 is a flow chart of a method for health optimization and medication adherence according to an embodiment of the present application. The flow chart 700 illustrated in FIG. 7 includes a data capture step where the product container/smart Pillbox captures user behavior. For example, the product container may capture user access to medicine compartments. In one embodiment, the microprocessor may send this data to the application software for storage into the database 710. The method may further include a data analytics and medication adherence step 720. The medication adherence step 720 may determine whether the medication is being taken according to the user's prescribed schedule.

If the user is not taking the medication according to the established medication schedule, the system may send notifications 730 to the user and or interested party via reminders. For example, the product container may begin with blinking an LED light to alert the user. In one embodiment, the product container may sound an alarm. In yet another embodiment the system may also send a text message to the user's device. If the user is taking the medication according to the established medication schedule, the system may store the user behavior in a data center 750 and/or send the information to a distributed data center 760. In one embodiment the user behavior records details of who accessed the product dispenser, when the access occurred, which medication was taken and from which compartment. Another step in the flow chart illustrated in FIG. 7 is the Post Notification Data Analytics Medication Adherence step 740. Again, a determination is made whether the user is taking the medication according to an established medication schedule. If the user is taking the medication according to the established medication schedule, then the user behavior may be recorded to the data center. If the user is not taking the medication according to the schedule, notifications and/or digital interventions may include the system documenting and recording potentially missed medication doses and the details thereof in the distributed data center 760.

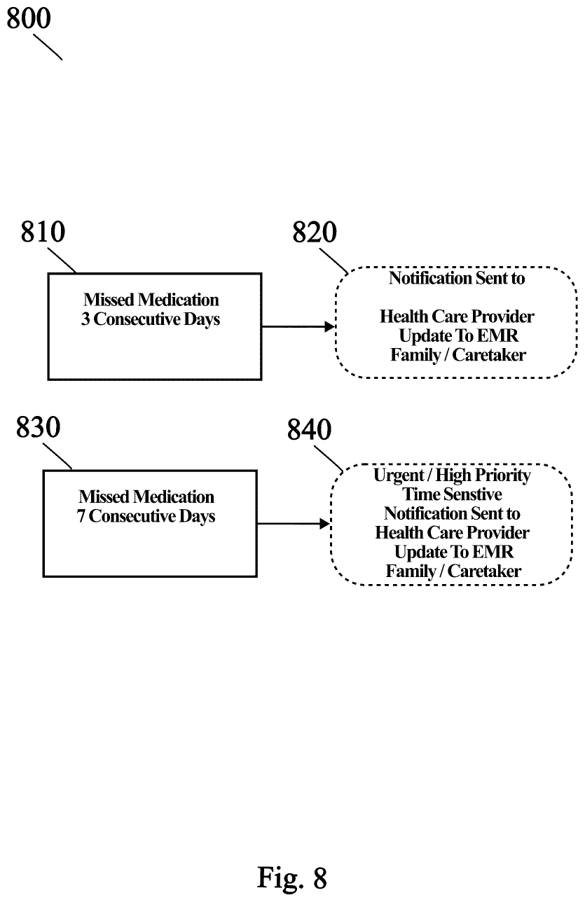

FIG. 8 is a flow chart of a method for health optimization and medication adherence according to an embodiment of the present application. The flow chart 800 illustrated in FIG. 8 Shows monitoring user adherence to a medication schedule and the corresponding interventions for extended non-compliance. For example, step 810 indicates a missed medication for three consecutive days. In this situation a notification 820 may be sent to a healthcare provider, an update may be made to an electronic medical record, and/or a notification may be sent to a family and/or a caretaker. Additionally, if a medication is missed for seven consecutive days 830, a phone call, text, e-mail may be sent to the user, a user's family, a user's physician, a nurse and/or other caregiver 840. The system may be able to distinguish between low risk medications and high risk medications. For example, if a patient is prescribed an over the counter pain medication like acetaminophen and the patient misses a dose, the system may trigger a single notification. However, if a blood pressure or an anti-seizure medication is prescribed for a patient who may also be a high risk of heart attack and/or seizure, the system may trigger an “urgent notification” with multiple notifications to the authorized user, the medical professionals, caretakers, first responders and/or the like. The system may also trigger urgent notifications when a predetermined number of medication doses are missed. This may depend on the patient's profile, the type of medication and the calculated risk of an adverse event.

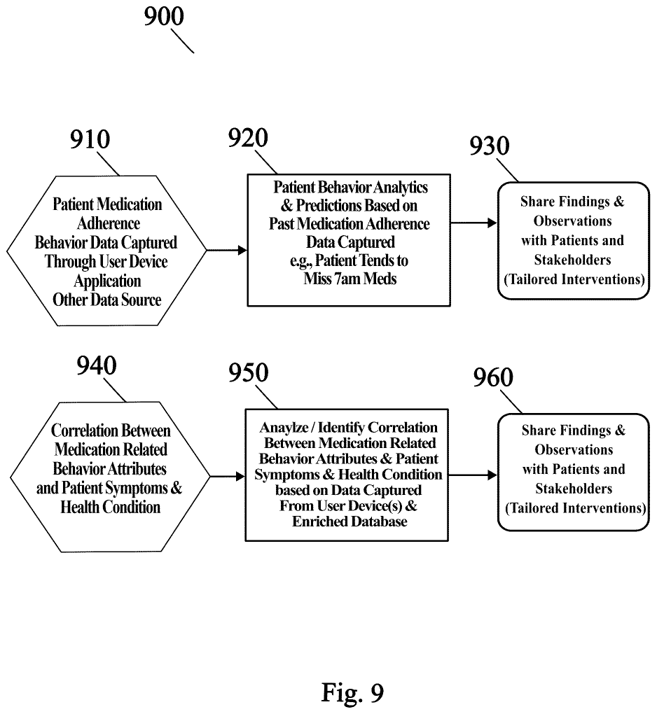

FIG. 9 is a flow chart of a method for health optimization and medication adherence according to an embodiment of the present application. The flow chart in FIG. 9 shows a method for leveraging machine learning and generative AI data. In one embodiment, machine learning and generative AI may provide analytics and/or predictions to suggest personalized interventions and or analysis of user information from an enriched database. According to one embodiment, user medication adherence behavior data 910 may be captured through a user device, application software and/or other sources.

A machine learning algorithm may be utilized in combination with patient behavior data to provide predictions based on past medication adherence captured by the product container, user device and/or application software 920. For example, the user's behavior analytics may capture a patient's trend or tendency to miss 7:00 AM or early morning medications. The findings and observations may be shared with the user and stakeholders 930. The flow chart further illustrates utilizing a machine learning algorithm to develop a correlation between medication related behavior attributes and patient symptoms and/or health condition 940. In one embodiment, the system may analyze and identify a correlation between medical medication related behavior attributes, patient symptoms and health conditions based on data captured from a user device and the enriched database 950. Similarly the method may share these findings and observations with a user and stakeholders 960.

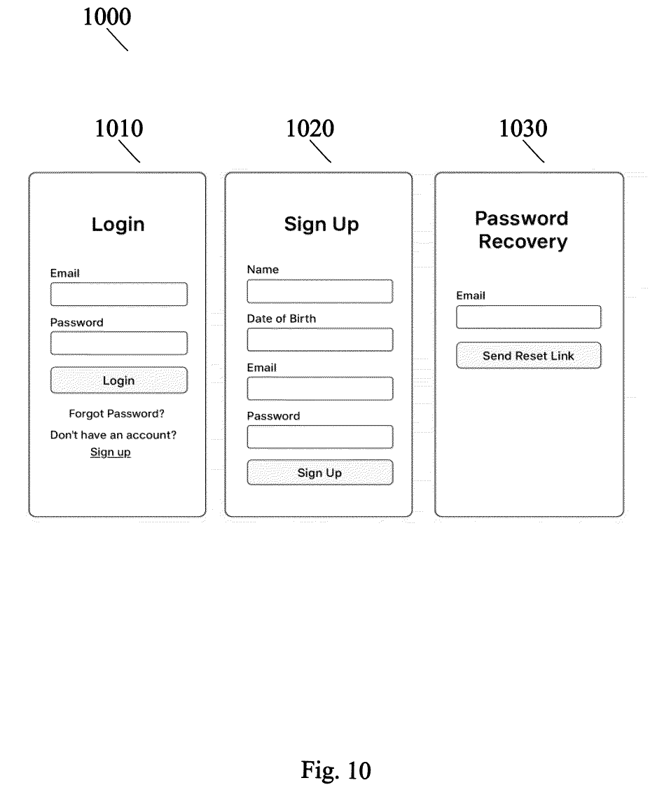

FIG. 10 is a user interface for a system for health optimization and medication adherence according to an embodiment of the present application. The user interface 100 illustrated in FIG. 10 includes a login screen 1010, a sign up screen 1020 and a password recovery screen. These screens show one approach to account creation however other methods of creating accounts may also be utilized. For example, one implementation may permit federated access or federated identity. In one embodiment, federated access is a relationship between three distinct parties. One party is the principal user, the individual trying to obtain access. The Identity Provider (IdP) is the party or system that actually owns the user's password and has already verified who they are. The Service Provider (SP) is the external application or resource the user wants to use. In one embodiment, the system and method for health optimization and medication adherence may act as the service provider allowing federated access from an identity provider. For example, a user may utilize their healthcare provider's software application as the Identity Provider. Similarly, a user may utilize their email provider as the Identity Provider. In other implementations, the system and method for health optimization and medication adherence may itself be the Identity Provider and the service may be the user's electronic medical records and/or healthcare provider's software application.

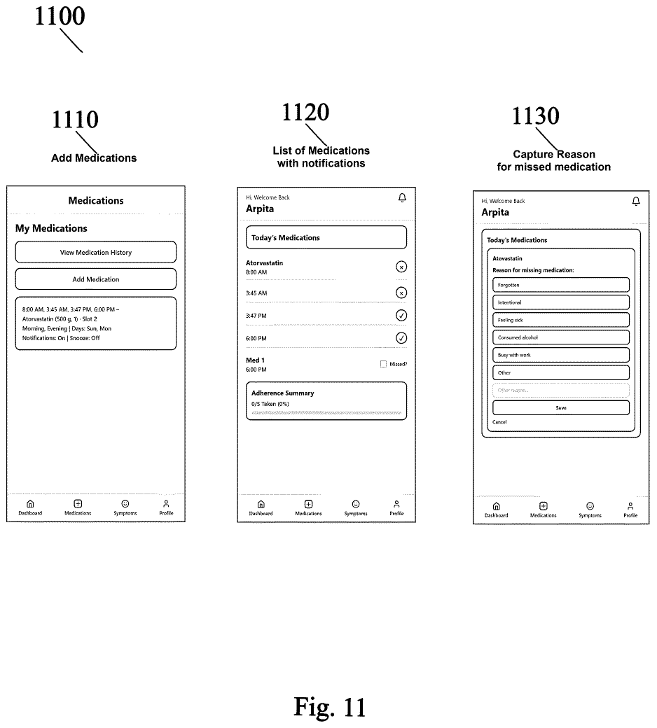

FIG. 11 is a user interface for a system for health optimization and medication adherence according to an embodiment of the present application. The user interface 1100 illustrated in FIG. 11 includes a My Medications interface 1110, a list of medications with notifications interface 1120 and a capture reason for missed medication interface 1130. My Medications interface 1110 may include a My Medications tab, a Medication History function, an add Medications tab and a medication schedule. The list of medications with notifications interface includes a list of today's medications and times. The interface allows a user to check off a scheduled medication dose as taken or missed. In one implementation, the interface may provide the user with a visual summary of their medication adherence. Medication adherence may be presented per medication. Medication adherence may also be calculated for one or more of the medications by day, week and/or monthly interval. The capture reason for missed medication interface 1130 may provide a prompt to the user for missing the medication. Once the prompt captures this data, the user data may be transmitted to the database/data center where it may be incorporated into the data analytics/machine learning algorithms to inform the generalized and/or tailored interventions.

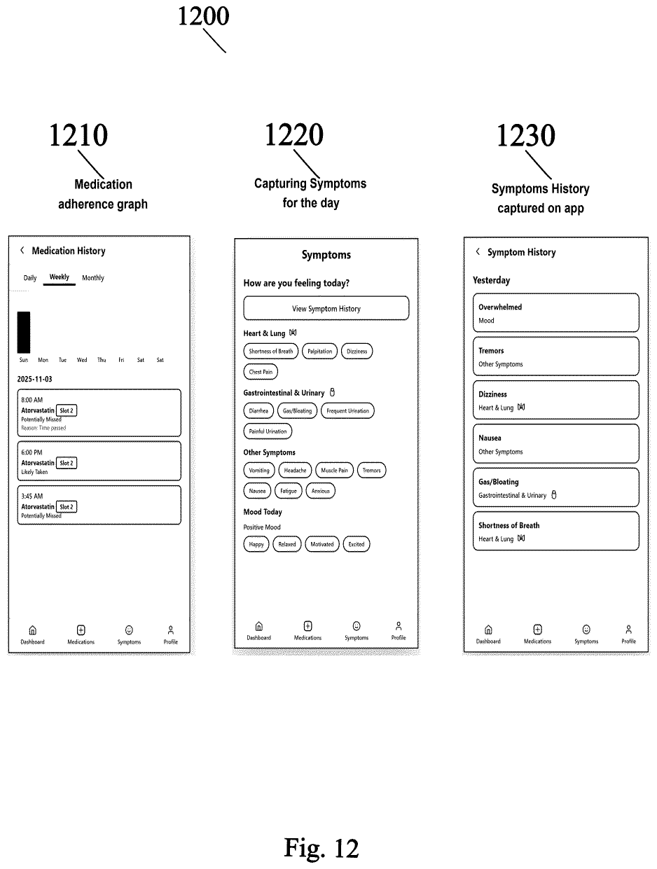

FIG. 12 is a user interface for a system for health optimization and medication adherence according to an embodiment of the present application. The user interface 1200 may include a medication adherence graph interface 1210, a symptom capture interface 1220 and a symptoms history interface 1230. The medication adherence graph interface may include a temporal selection including day, weekly, monthly and yearly interval. The medication adherence graph may present medication adherence via a bar graph, line graph, pie chart, and the like. The medication adherence graph may also show upcoming medications, dosages and schedule.

The symptom capture interface 1220 interface may present a general prompt, e.g., “How are you feeling today?” and a symptom history function. The symptom capture interface also shows more specific symptom prompts involving 1) Heart & Lung 2) Gastrointestinal & Urinary 3) Other Symptoms and 4) Mood. In one implementation, the symptom capture interface may utilize the list of user medications to prompt the user with tailored symptoms commonly associated with the user's prescribed medications. In one implementation, symptoms may be passively captured by other devices, including wearable devices, and stored in the database/data center for additional processing. For example, a user treating a skin condition may upload photos of the affected skin area which may illustrate an increase or decrease in inflammation depending on the efficacy of the prescribed medication.

The symptoms history interface 1230 may present the user with a list of symptoms previously reported. The history interface may be filtered temporally by day, week, month and year. Additionally, the user may select a period of time beginning at the start of a new medication(s) and/or a change in dosage. Similarly, the period of time may also include the date of discontinuance and or tapering of a medication.

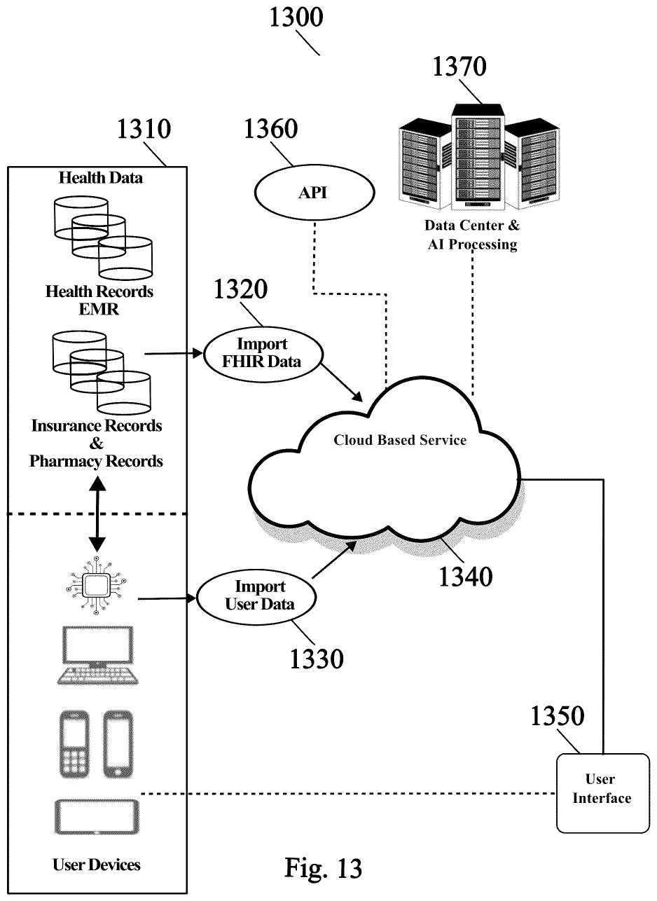

FIG. 13 is a network diagram for a system for health optimization and medication adherence according to an embodiment of the present application. The network 1300 depicted in FIG. 13 includes a plurality of data sources 1310. The plurality of data sources may include, but is not limited to, health data (Electronic Medical Records, Insurance Records and Pharmacy Records, etc.) as well as data captured by user devices. The health data may be imported via a Fast Healthcare Interoperability Resources standard (“FHIR”) and via sharing user data with the application software. These imports may be sent over a network to a cloud based service 1340. The data may be processed or accessed via an API 1360 and/or user interface 1350. In one implementation, the health data may be sent to a data center for storage and processing by one or more artificial intelligence or machine learning algorithm(s) 1370.

In one embodiment, the artificial intelligence/machine learning algorithms may include large language models. A Large Language Model (LLM) is a sophisticated form of artificial intelligence that operates on the principles of deep learning, specifically utilizing transformer-based neural network architectures. These models are characterized by their vast number of parameters, often scaling to billions or even trillions, which allow them to process, understand, and generate human language with remarkable proficiency. The core mechanism of an LLM is rooted in the transformer architecture, which employs self-attention mechanisms to weigh the importance of different words in a sentence or sequence, enabling the model to capture contextual relationships over long distances within the text. This architecture consists of multiple layers of attention and feedforward neural networks, which process the input text in parallel, significantly improving efficiency and learning capacity compared to previous sequential models.

LLMs may be trained through a process called unsupervised learning on massive datasets compiled from a wide array of textual sources across the internet and other digital repositories. In the proposed system, the training data may include health data 1310 and training data 1410. During training, the model is exposed to large volumes of text, learning to predict the next word in a sentence given the words that precede it. This prediction task forces the model to learn linguistic patterns, grammar, semantics, and even factual information to some extent. The training process involves adjusting the weights of the neural network to minimize the difference between the model's predictions and the actual text, a process facilitated by backpropagation and optimized through techniques such as gradient descent.

Once trained, LLMs can generate text, answer questions, summarize content, translate languages, and perform other language-related tasks by generating probabilities for the next word or sequence of words based on the input provided. The model selects the most probable next word repeatedly, constructing sentences and paragraphs that align with the patterns it learned during training. The effectiveness of an LLM is significantly influenced by its parameter count and the diversity and quality of its training data. However, these models also face challenges, including biases inherited from training data, difficulties with ensuring factual accuracy, and the computational resources required for training and inference. Advances in AI, including novel training techniques, model architectures, and ethical guidelines, are ongoing to address these challenges and enhance the capabilities and applications of LLMs in technology and society.

A context window of a Large Language Model (LLM) refers to the maximum span of text (in terms of tokens, which can be words or sub words) that the model can consider at one time when making predictions or generating text. This limitation is inherent to the model's architecture, particularly in transformer-based models, which are commonly used in LLMs. The context window size is a critical factor in determining the model's ability to understand and generate coherent and contextually relevant language outputs.

The input text is tokenized or broken down into tokens, which can be words, parts of words, or punctuation marks, depending on the tokenization scheme used by the model. Each token is then converted into a numerical representation (embedding) that the model can process. Transformer models may also use an attention mechanism to weigh the importance of each token in the context window relative to the token currently being processed. This mechanism allows the model to consider the entire context when predicting the next token, making the size of the context window crucial for capturing long-range dependencies and understanding complex sentences.

In one embodiment, the context window may receive immediate context or the most recent words or tokens that directly precede the point of prediction. The context window may also take in relevant information or information relevant to the current sentence or idea being processed, such as subject names, key actions, and attributes, even if they appeared earlier in the text. The context window may also utilize cross-sentence dependencies or relationships and dependencies that span multiple sentences but fall within the context window, enabling the model to maintain coherence over longer stretches of text.

The AI model may also be fine-tuned. Fine-tuning in the context of machine learning, particularly with Large Language Models (LLMs), is a process where a pre-trained model is further adjusted or trained on a smaller, specific dataset to adapt its knowledge and capabilities to a particular task or domain. This technique leverages the general understanding and the extensive base of knowledge that the model has acquired during its initial, extensive training on vast and diverse datasets. The goal of fine-tuning is to make the model more relevant and effective for tasks that may not have been well-represented in the original training data, or to improve its performance on specific types of input.

The outputs of a Large Language Model (LLM) can be diverse, ranging from plain text generation to more complex interactions like generating code, executing function calls. For example, some LLMs may be expanded significantly with advancements in model architecture, training methodologies, and integration techniques, allowing them to not only generate text but also to interact with external systems (smart homes/smart home devices, databases, and programming interfaces.

The LLM may include function calling or executing code. LLMs, especially those integrated with coding capabilities or trained explicitly on programming languages, can understand, and generate executable code snippets based on natural language descriptions. This allows them to interact with APIs and external systems. For example, an advanced application of LLMs involves generating outputs that include calls to external APIs (Application Programming Interfaces) or systems. This enables the LLM to retrieve information from, or send commands to, databases, web services, and other software applications, effectively allowing the model to interact with the outside world. Implementing function calling with LLMs involves sophisticated integration between the language model and the target functions or services. This requires careful handling of security, privacy, and error management to ensure that interactions are safe and reliable. Moreover, the model must be capable of understanding when and how to make function calls appropriately within the context of its output, which may involve complex reasoning and understanding of the user's intent.

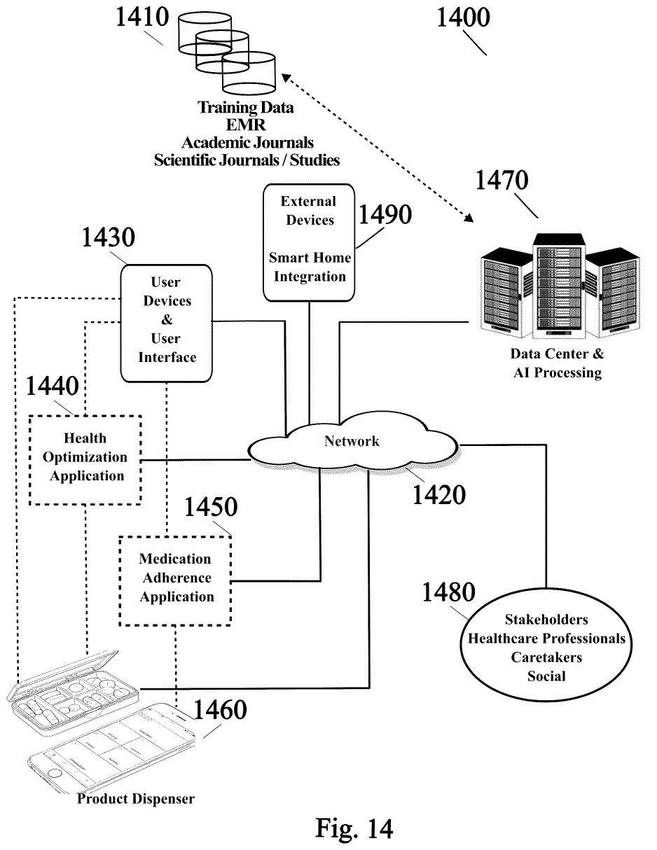

FIG. 14 is a network diagram for a system for health optimization and medication adherence according to an embodiment of the present application. The network diagram 1400 includes a set of training data 1410 including electronic medical records, academic journals, scientific journals and studies. The training data may also include user behavior data previously captured on other systems and/or devices. The training data may be received and stored at data center 1470 where it may be used to train one or more machine learning models. The data center is connected to a network 1420. The network is in turn in communication with one or more user devices/user interfaces 1430, a health optimization application 1440, a medication adherence application 1450, a product container 1460 and one or more stakeholders 1480. In one implementation, the user device/user interface may be in communication with the product container through the two applications 1440, 1450 without network connectivity. For example, the user device may connect to the product container via Bluetooth technology. The system may further integrate with external devices and integrate with an authorized user's smart home 1490.

The external devices may include climate/temperature devices such as thermostats, air conditioners and heaters, air purifiers, humidifiers, dehumidifiers, and ceiling fans. Smart home devices may also include lighting controls such as smart light bulbs, smart switches, plugs, outlets, and power strips that allows the system to automate schedules and control devices remotely. Entertainment systems, smart TVs, streaming devices, smart speakers may also be integrated to the health optimization platform. Smart refrigerators, ovens, microwaves, dishwashers, coffee makers can all be integrated as well.

The health optimization and medication adherence application may work separately or in combination with overlapping features and access to data. In one implementation, the applications may analyze the user behavior information including comparing it to, for example, one or more of: the established medication schedule, user provided instructions regarding the types and timing of reminders and alerts, and patient information obtained from Electronic Medical Record (EMR) systems. The system may determine whether, when and what kind of messages in the form of reminders, alerts, or combinations thereof to issue to the user. Messages may be bi-directional or unidirectional depending on the user's needs. For example, the app may send reminders before the scheduled medication time and again at the exact medication time. If the user does not open the pillbox (as determined using the sensors), the medication adherence application (or product container equipped with a speaker) may further issue an alarm at regular intervals, until the medication is taken. The frequency, duration, and sound of the alarm may be customizable.

In one implementation, a large language model (LLM) may be trained on training data 1410. The language model will use curated medical literature, clinical guidelines, drug reference sources, and peer-reviewed research as reference material to support medication adherence and patient education features within the medication adherence ecosystem. The system is designed to provide informational and contextual assistance related to medication use and adherence behavior, while supporting communication among patients, caregivers, and healthcare providers about the medication adherence and its possible effects. In certain embodiments, the language model may learn expected medication effects, common tolerability patterns, and known associations between medications and frequently reported symptoms from validated literature sources. When combined with adherence data generated within the ecosystem, the system may identify potential correlations between medication use patterns and user-reported symptoms. For example, if a patient taking metformin reports gastrointestinal symptoms such as diarrhea, the system may recognize this as a commonly reported effect and provide educational context while enabling appropriate visibility to designated stakeholders.

The functionality may support adherence-focused workflows, including providing educational information to patients about commonly observed medication effects to reduce unnecessary discontinuation of therapy. The workflows may also include encouraging patients to report symptoms or medication concerns within the application. This may be accomplished through prompts and incentive based measures. In one implementation, the system may alert caregivers or designated providers when adherence changes or symptom reports may affect medication continuation. The system may further provide a graphical user interface provider dashboard assisting providers to visualize adherence patterns alongside symptom reports to support informed patient discussions. The system may also support timely, patient-centered interventions aimed at maintaining adherence while addressing tolerability concerns.

In one implementation, the system for health optimization and medication adherence may include a data analytics module. The data analytics module may receive data regarding user interactions with the product container, user behavior data tracked by the health optimization application or the medication adherence application, user health data in the form of wearable device health data and/or health records. The data analytics module may process the data via a processor on the product dispenser, user device, and/or in the data center over the network. The applications may analyze the aforesaid information and, based on that information, provide users, doctors, and caregivers, with accurate information about potentially missed medication doses.

In one embodiment, the data center and AI processing system 1470 may utilize machine learning to provide services such as a medication schedule adherence predictive service tailored to adherence behavior and which is operated by the microprocessor, the app, cloud based service or a combination thereof. The AI-Powered Medication Schedule Adherence Predictive Service uses machine learning and user behavior information, previously collected as well as collected on an ongoing basis, to predict when a user is likely to miss a dose based on past behavior, mood logs, activity levels, time of day, etc. and preemptively notify caregivers or adapt reminders and suggest tailormade personalized interventions.

In one implementation, system and method for health optimization and medication adherence may include a Non-Compliance Remediation Algorithm which is performed by the microprocessor, the application(s), a cloud based service or a combination thereof. The non-compliance remediation algorithm may provide remediation features such as reaching out (e.g., notifying by email, text, etc.) to friends or family members of the user to check on and assist with medication administration compliance according to the established medication schedule. Alternatively, or in addition to friends or family members, a nurse or other home care provider service may be added as a potential source of compliance assistance and receive notifications according to the Non-Compliance Remediation Algorithm. Additionally, the application(s) may also provide educational information, such as webpages, voice messages, emails, and other communication methods, to users on the consequences of missed medications and may even offer incentives for compliance.

The system and method for health optimization and medication adherence may include a gamified adherence program, which may be helpful to engage users such as children, elderly, memory care patients, and other cognitively impaired patients. The gamified adherence program provides a behavioral reinforcement system integrated with physical medication control, for example, using a touchscreen or mobile app that gamifies medication adherence, offering rewards, visuals, or AI companions.

To gamify adherence, we can introduce AI driven dynamic difficulty adherence goals, where the system adjusts adherence challenges based on users'medication behavior patterns. It starts with easier goals for low adherence users and gradually increases expectations as habits improve. A machine learning model dynamically modifies adherence challenges based on predicted behavioral risk. In addition, we introduce Smart Recovery Mode after missed doses. Instead of penalizing missed doses, the system activates a Recovery Mode, offers short term achievable streaks, and provides motivation to restart adherence. This prevents dropout after streak breaks and represents adaptive recovery gamification triggered by adherence disruption. We also incorporate Caregiver and Family Celebration Triggers. When a user reaches adherence milestones, caregivers receive celebration prompts, and encouragement messages or rewards are triggered to reinforce positive behavior. This represents adherence milestone detection triggering social engagement workflows. Finally, a Personalized Motivation Engine learns what motivates each user, such as rewards, messages, competition, or family encouragement, and delivers tailored motivational triggers accordingly. This functions as a machine learning optimized motivational reinforcement system.

A gamified adherence system may incorporate a habit anchoring engine that utilizes artificial intelligence to associate medication intake with a user's existing daily routines and environmental interactions, including activities such as meal preparation, morning or bedtime routines, or use of household devices, and may further integrate with smart home or connected IoT systems to conditionally modify or enable environmental settings based on adherence status. Upon confirmation of scheduled medication intake through the medication container, biometric verification, or sensor-based detection, the system may activate or restore preferred environmental functions, such as enabling a coffee machine or kitchen appliance following completion of a morning dose, initiating preferred music, news briefings, or lighting scenes through smart speakers after medication intake, or unlocking normal operation of entertainment or comfort devices. Conversely, delayed or missed medication events may trigger configurable reminder actions, such as temporary playback of reminder audio through smart speakers, display notifications on connected home screens, or postponement of selected convenience functions until adherence is confirmed. These interactions may be customizable by the user or caregiver to provide positive reinforcement or gentle behavioral prompts, thereby embedding medication routines within everyday environmental cues to promote engagement, habit formation, and sustained adherence.

While the foregoing invention has been described in some detail for purposes of clarity and understanding, it will be appreciated by one skilled in the art, from a reading of the disclosure, that various changes in form and detail can be made without departing from the true scope of the invention.

1. A system for health optimization and medication adherence, comprising:
a product container capable of holding one or more products for dispensing to an authorized user;
a biometric sensor sensing one or more biometric attributes to determine access to a product container compartment;
a health optimization application tracking interactions with the product container to determine a medication adherence profile associated with the authorized user account; and
an adherence application transmitting one or more messages to the authorized user account.

2. The system for health optimization and medication adherence of claim 1, wherein the product container includes a base, one or more product compartments and a lid.

3. The system for health optimization and medication adherence of claim 2, wherein the one or more product compartments include one or more product sensors configured to sense a product count, a change in product count and whether one or more products is present in a given product compartment.

4. The system for health optimization and medication adherence of claim 2, wherein the lid includes a locking mechanism and a lid sensor configured to sense if the lid is open or closed.

5. The system for health optimization and medication adherence of claim 4, further comprising:
a processor and a memory in operable communication with a plurality of sensors including, the biometric sensor, compartment sensor and lid sensor; wherein the processor receives data from the plurality of sensors and stores the data in the memory.

6. The system for health optimization and medication adherence of claim 5, further comprising:
a HIPPA compliant database receiving, storing and transmitting electronic medical records associated with the authorized user;
a user interactions database receiving, storing and transmitting user interactions with the product dispenser;
a user health database receiving, storing and transmitting user health data from one or more user devices.

7. The system for health optimization and medication adherence of claim 6, further comprising:
an enriched database combining the HIPPA compliant database, the user interactions database and the user health database.

8. The system for health optimization and medication adherence of claim 7, further comprising:
a data analytics application analyzing information in the enriched database to determine adherence with a product schedule.

9. The system for health optimization and medication adherence of claim 8, further comprising:
a messaging application configured to transmit one or more messages based, at least in part, on an output received from the data analytics application.

10. The system for health optimization and medication adherence of claim 9, wherein the one or more messages includes an e-mail, a text message, an audio message, a visual message, a status light and a haptic feedback.

11. The system for health optimization and medication adherence of claim 10, further comprising:
a user interface presenting the product schedule and the one or more messages.

12. The system for health optimization and medication adherence of claim 11, further comprising:
a machine learning algorithm receiving data from the enriched database, the machine learning algorithm developing a baseline behavioral profile for the authorized user, the baseline behavioral profile indicating a user's adherence to the product schedule.

13. The system for health optimization and medication adherence of claim 12, wherein the machine learning algorithm analyzes information stored in the enriched database and generates a predictive profile indicating likelihood of adherence.

14. The system for health optimization and medication adherence of claim 13, wherein, based on the predictive profile, the machine learning algorithm generates one or more interventions designed to increase adherence to the product schedule.

15. The system for health optimization and medication adherence of claim 14, wherein the one or more interventions is a generalized intervention.

16. The system for health optimization and medication adherence of claim 14, wherein the one or more interventions is a tailored intervention.

17. The system for health optimization and medication adherence of claim 14, wherein the one or more interventions includes a suggested modification to the product schedule.

18. The system for health optimization and medication adherence of claim 14, wherein the one or more interventions transmitting an intervention message to one or more user devices wherein the one or more interventions includes:
transmitting the intervention message to the one or more user devices includes transmitting the intervention message to a mobile phone; and
transmitting the intervention message to the product dispenser.

19. The system for health optimization and medication adherence of claim 14, wherein the machine learning algorithm analyzes an effect of medication adherence by comparing a user's health data received from one or more user devices, the user's health data including one or more vital signals, one or more symptoms.

20. The system for health optimization and medication adherence of claim 18, wherein the one or more interventions includes transmitting an intervention message to one or more stakeholders.
